(19) United States
(12) Patent Application Publication
(10) Pub. No.: US 2010/0004704 A1
(43) Pub. Date: Jan. 7, 2010

(54) METHOD FOR TREATING A CARDIAC RHYTHM DISTURBANCE

(75) Inventors: Todor N. Mazgalev, Cleveland, OH (US); Zhang Youhua, Beachwood, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/433,019

(22) Filed: Apr. 30, 2009

Related U.S. Application Data
(60) Provisional application No. 61/049,782, filed on May 2, 2008.

(51) Int. Cl. A61N 1/362

(52) U.S. Cl. 607/3

(57) ABSTRACT

A method is provided for treating a subject. One step of the method includes performing cardiac surgery on the subject. Next, a cardiac drug is administered to the subject. An electric current is then applied to a cardiac fat pad of the subject to reduce an adverse effect caused by the cardiac drug.

### RELATED APPLICATION

This application claims priority from U.S. Provisional Application No. 61/049,782, filed May 2, 2008, the subject matter of which is incorporated herein by reference.

### TECHNICAL FIELD

The present invention relates generally to a method for treating heart conditions, and more particularly to a method for treating a cardiac rhythm disturbance following cardiac surgery and cardiac drug administration.

### BACKGROUND OF THE INVENTION

Performance of cardiac surgery is a delicate and invasive procedure. The majority of epicardial bypass graft surgeries and open-heart procedures, for example, require temporary arrest of the heart to allow a surgeon to accomplish the required task without interference from heart movement. The taxing nature of cardiac surgery often requires patients to undergo temporary inotropic support.

Pharmacologic inotropic agents enhance myocardial contractility and fall into two broad categories: sympathomimetics, such as epinephrine (adrenaline), norepinephrine (noradrenaline), dobutatnine, isoproterenol, salbutamol, salmeterol, terbutaline, isoproterenol, phenylephrine, ephedrine, and dopamine; and phosphodiesterase inhibitors, such as milrinone and amrinone. Each of these agents, while increasing the inotropic state of the heart, has limitations that restrict the doses that can be given intravenously and often necessitate infusion of additional agents to counteract side effects.

One important consideration when using inotropic agents is that these agents treat all vascular beds when administered systemically. Consequently, systemic side effects of sympathomimetics include potential renal and cerebral vasoconstriction, as well as pulmonary artery hypertension. Other undesired side effects include excess tachycardia and electrical irritability of the heart.

### SUMMARY OF THE INVENTION

According to one aspect of the present invention, a method is provided for treating a subject. One step of the method includes performing cardiac surgery on the subject. Next, a cardiac drug is administered to the subject. An electric current is then applied to a cardiac fat pad of the subject to reduce an adverse effect caused by the cardiac drug.

According to another aspect of the subject invention, a method is provided for treating a subject. One step of the method includes performing cardiac surgery on the subject. Next, a positive inotropic drug is administered to the subject. An electric current is then applied to a sinus node fat pad of the subject. The electric current is configured so as to increase vagal activity in the subject and thereby reduce sinus tachycardia caused by the positive inotropic drug.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a process flow chart illustrating a method for treating a subject suffering from a cardiac rhythm disturbance according to the present invention;

FIG. 2A is a cross-sectional view of a human heart;

FIG. 2B is a perspective view showing the right side of the heart in FIG. 2A; and

FIG. 2C is a perspective view showing the posterior view of the heart in FIG. 2A.

### DETAILED DESCRIPTION

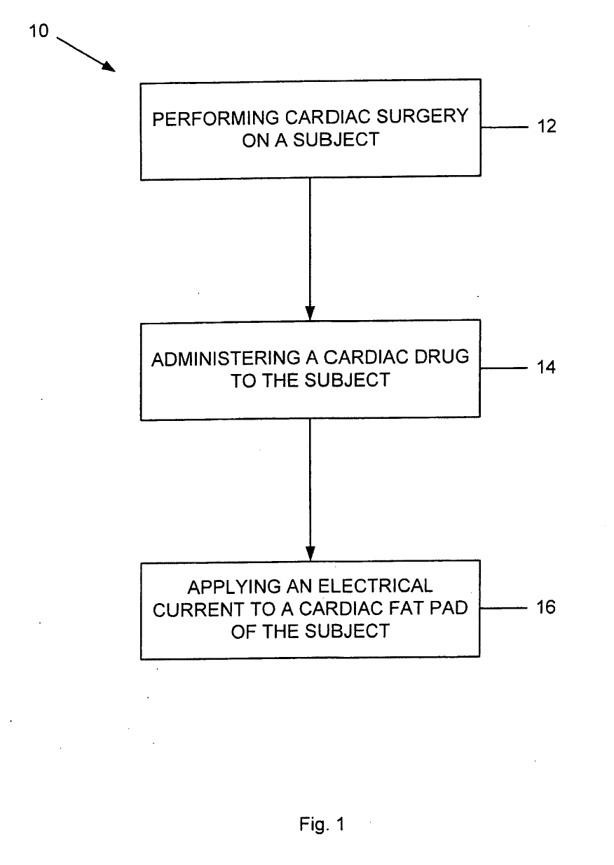

The present invention relates generally to a method for treating heart conditions, and more particularly to a method for treating a cardiac rhythm disturbance following cardiac surgery and cardiac drug administration. As representative of the present invention, FIG. 1 illustrates a method 10 for treating a subject suffering from a cardiac rhythm disturbance. Although the present invention is described in terms of treating sinus tachycardia following open-heart surgery, it will be appreciated that the present invention can treat other heart conditions, such as those listed below.

Unless otherwise defined, all technical terms used herein have the same meaning as commonly understood by one of ordinary skill in the art to which the present invention pertains.

In the context of the present invention, the term “cardiac surgery” can refer to surgery on the heart and/or the great vessels, which is typically performed by a cardiac surgeon. Non-limiting examples of cardiac surgeries include closed-heart surgery, minimally invasive heart surgery, beating-heart surgery, open-heart surgery, and percutaneous heart surgery.

As used herein, the term “cardiac drug” can refer to any pharmaceutically active agent used to treat a heart condition or cardiac rhythm disturbance. Examples of cardiac drugs include, but are not limited to, growth factors, polynucleotides encoding growth factors, angiogenic agents, calcium channel blockers, antihypertensive agents, antimitotic agents, inotropic agents, anti-atherogenic agents, anti-coagulants, β-blockers, anti-arrhythmic agents, anti-inflammatory agents, sinus rhythm management agents, vasodilators, thrombolytic agents, cardiac glycosides, antibiotics, antiviral agents, antifungal agents, agents that inhibit protozoans, angiotensin converting enzyme inhibitors, brain natriuretic peptide, antineoplastic agents, and steroids.

As used herein, the term “adverse effect” can refer to any condition in which the heart does not function normally, such as abnormal cardiac electrical conductivity that results in an irregular heartbeat or dysfunctional cardiac rhythm. Examples of dysfunctional cardiac rhythms include, but are not limited to, premature atrial contractions or supraventricular contractions, supraventricular tachycardia or paroxysmal SVT, atrial fibrillation, atrial flutter, ventricular fibrillation, ventricular tachycardia, premature ventricular contractions, sinus tachycardia, sinus bradycardia, and Wolf-Parkinson-White syndrome.

As used herein, the term “heart condition” refers to a wide range of abnormalities and/or diseases of the heart, coronary vasculature, or blood vessels surrounding the heart, including underlying conditions, such as ischemia, atherosclerosis or coronary artery disease, embolism, congenital heart defects, anemia, lung disease, and abnormal stimulation (e.g., sympathomimetic abuse), hypertension (e.g., systemic hypertension, primary and secondary hypertension, pulmonary hypertension), chronic obstructive pulmonary disease, restrictive lung disease, pulmonary embolism, morbid obesity, valvular disease (e.g., mitral valve disease, aortic valve disease, tricuspid valve disease, and pulmonary valve disease), heart muscle disease (e.g., ischemic cardiomyopathy, dilated cardiomyopathy, hypertensive cardiomyopathy, hypertrophic cardiomyopathy, restrictive cardiomyopathy, and specific heart muscle disease resulting from cardiac infection), neuromuscular disease, storage disorders, infiltration disorders, immunologic disorders, pericardial disease, rheumatoid heart disease, neoplastic heart disease (e.g., primary cardiac tumors), coronary vasospasm (e.g., drug-induced vasospasm), cardiac trauma, genetic or hereditary predisposition that may manifest as angina (e.g., stable angina, unstable angina, mixed angina, and Prinzmetal's variant angina), myocardial infarction, chronic ischemic heart disease, and sudden cardiac death.

A brief discussion of the cardiac anatomy and physiology is provided to assist the reader with understanding the present invention. The automatic nervous system (ANS) regulates “involuntary” organs, while the contraction of voluntary (skeletal) muscles is controlled by somatic motor nerves. Examples of involuntary organs include respiratory and digestive organs, as well as blood vessels and the heart. Often, the ANS functions in an involuntary, reflexive manner to regulate glands, muscles in the skin, the eyes, stomach, intestines, bladder, cardiac muscles, and muscles surrounding blood vessels, for example.

The ANS includes the sympathetic nervous system (SNS) and the parasympathetic nervous system (PNS). The SNS is affiliated with stress and the “fight-or-flight response” to emergencies. Among other effects, the “fight-or-flight response” increases blood pressure and heart rate, thereby increasing skeletal muscle blood flow and decreasing digestion to provide energy for “fighting or fleeing.” The PNS is affiliated with relaxation and the “rest and digest response” which, among other effects, decreases blood pressure and heart rate, and increases digestion to conserve energy. The ANS maintains normal internal function and works with the somatic nervous system.

Electrically stimulating the SNS and PNS can have a number of physiological effects. For example, stimulating the SNS dilates the pupils, reduces saliva and mucus production, relaxes the bronchial muscle, reduces peristalsis of the stomach, increases the conversion of glycogen to glucose by the liver, decreases urine secretion by the kidneys, and closes the sphincter of the bladder. Stimulating the PNS constricts the pupils, increases saliva and mucus production, contracts the bronchial muscle, increases secretions and motility in the stomach and large intestine, increases digestion in the small intestine, increases urine secretion, and relaxes the sphincter of the bladder.

The functions associated with the SNS and PNS can be integrated with each other. Thus, indiscriminate stimulation of the SNS and/or PNS to achieve a desired response, such as vasodilation, in one physiological system may also result in an undesired response in another physiological system. As described in more detail below, the present invention provides a targeted approach to modulating vagal activity and thereby effecting cardiac function to avoid such effects.

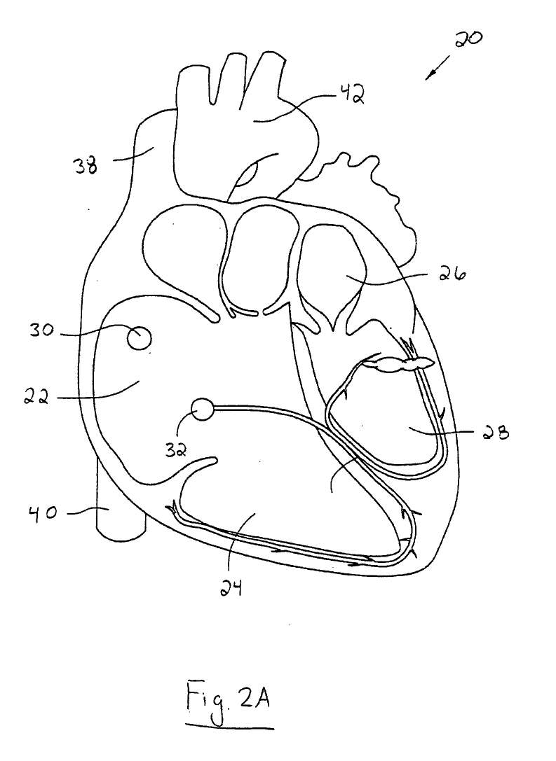

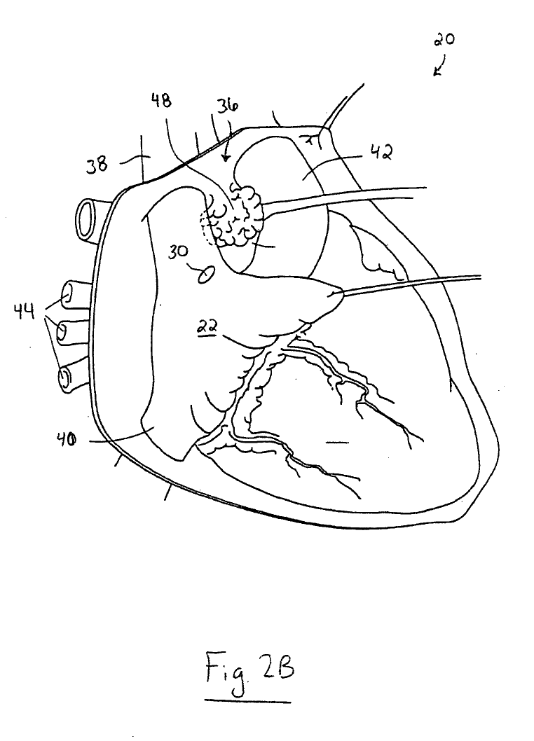

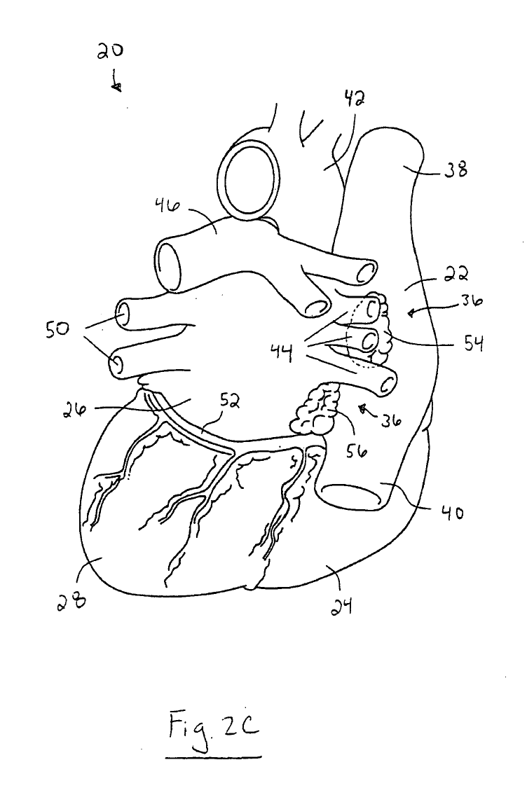

FIGS. 2A-C illustrate a human heart 20. The heart 20 includes a right atrium 22, a right ventricle 24, a left atrium (LA) 26, and a left ventricle 28. The heart 20 also includes a sinoatrial (SA) node 30 and an atrioventricular (AV) node 32. The SA node 30 comprises a cluster of cells in the right atrium 22 that generates electrical impulses. The AV node 32 comprises a cluster of cells situated in the center of the heart 20 between the atria 22 and 26 and the ventricles 24 and 28.

FIG. 2A illustrates the cardiac conduction system that controls heart rate. This system generates and conducts electrical impulses throughout the myocardium to stimulate cardiac constriction. The cardiac conduction system includes the SA node 30 and the AV node 32.

The ANS controls firing of the SA node 30 to trigger the start of the cardiac cycle. The electrical impulses generated by the SA node 30 are propagated between myocardial cells until the impulses reach the AV node 32. The AV node 32 functions as an electrical relay station between the atria 22 and 26 and the ventricles 24 and 28, such that electrical signals from the atria must pass through the AV node to reach the ventricles. The AV node 32 slows the electrical current before the signal is permitted to pass through the ventricles 24 and 28, thereby allowing the atria 22 and 26 to fully contract before the ventricles are stimulated. After passing the AV node 32, electrical impulses travel to the ventricles 24 and 28 along Purkinje fibers 34 embedded in the inner ventricular walls of the heart 20.

FIGS. 2B-C show the cardiac fat pads 36 of the heart 20. FIG. 2B shows the right atrium 22, right ventricle 24, the SA node 30, the superior vena cava (SVC) 38, the inferior vena cava (IVC) 40, the aorta (AO) 42, the right pulmonary veins 44, and the right pulmonary artery 46. FIG. 2B also shows a cardiac fat pad 36, referred to herein as the SVC-AO fat pad 48, located between the superior vena cava 38 and the aorta 42. FIG. 2C shows the left atrium 26, the left ventricle 28, the right atrium 22, the right ventricle 24, the superior vena cava 38, the inferior vena cava 40, the aorta 42, the right pulmonary veins 44, the left pulmonary veins 50, the right pulmonary artery 46, and the coronary sinus 52. FIG. 2C also shows a cardiac fat pad 36, referred to herein as the SA node (SN) fat pad 54, located proximate to a junction between the right atrium 22 and the right pulmonary veins 44. Additionally, FIG. 2C shows a cardiac fat pad 36, referred to herein as the IVC-LA fat pad 56, located proximate to or at the junction of the inferior vena cava 40 and the left atrium 26.

The SVC-AO fat pad 48 functions as a “head station” of vagal fibers (not shown) projecting to the right and left atria 22 and 26, the IVC-LA fat pad 56, and the SN fat pad 54. The portion of the ANS that regulates heart rhythm includes a number of ganglionated fat pads, i.e., the SVC-AO fat pad 48, the IVC-LA fat pad 56, and the SN fat pad 54. Parasympathetic ganglia in these cardiac fat pads 36 exert important effects on chronotropy, dromotropy, and inotropy. For example, cardiac rate, AV conduction, and contractility are mediated through ganglia located in these cardiac fat pads 36.

Disruption of neural activity in the cardiac fat pads 36 can cause significant heterogeneity of repolarization, and tends to result in atrial arrhythmias. An intrinsic cardiac neuronal network is important to both intracardiac and extracardiac integration of autonomic cardiac function. Unfortunately, this cardiac neuronal network can be damaged, thus adversely affecting the autonomic balance. For example, myocardial ischemia can compromise the function of neurons embedded with the cardiac fat pads 36, diabetic neuropathy can affect intrinsic cardiac innervation, and surgery may sever or otherwise damage a portion of the cardiac neural network.

FIG. 1 is process flow diagram illustrating an aspect of the present invention. In FIG. 1, a method 10 is provided for treating a subject suffering from a cardiac rhythm disturbance. At 12, one step of the method 10 includes performing cardiac surgery on the subject. Although the term “subject” as used herein typically refers to a human subject, it will be appreciated that the term can also include any warm-blooded organism including, but not limited to, pigs, rats, mice, dogs, goats, sheep, horses, monkeys, apes, rabbits, cattle, etc. The type of cardiac surgery performed will depend on the clinical need(s) of the subject. For example, a subject suffering from coronary artery disease (CAD) may require coronary artery bypass graft (CABG) surgery. Alternatively, a subject suffering from a regurgitant mitral valve may require minimally-invasive valve replacement surgery.

In an example of the method 10, a subject may require an open-chest procedure, such as CABG to treat CAD. Approaches to performing CABG are well known in the art. Briefly, an incision (not shown) is made down the middle of the chest, which is followed by a stemotomy. The heart 20 is cooled with iced salt water, while a preservative solution is injected into the cardiac arteries. Before bypass surgery can take place, a cardiopulmonary bypass (not shown) must be established. Plastic tubes (not shown) are placed in the right atrium 22 to channel venous blood out of the body for passage through a plastic sheeting (e.g., membrane oxygenator) (not shown) and into a heart-lung machine (not shown). Oxygenated blood is then returned to the body, and the aorta 42 is clamped-off to maintain a bloodless field and allow bypasses to be connected to the aorta.

A saphenous vein (not shown) may be used to bypass an arterial blockage (not shown). Bypass grafting involves sewing the graft vessel to a coronary artery (not shown) just beyond the arterial narrowing or blockage. The other end of the graft is attached to the aorta 42. Chest wall arteries, particularly the left internal mammary artery (not shown), may also be used as bypass grafts. Typically, the aorta 42 is clamped-off for about 60 minutes, whereafter the subject is supported by cardiopulmonary bypass for about 90 minutes. At the end of surgery, the sternum is wired together and the chest incision is sewn closed. Plastic chest tubes (not shown) are left in place to allow drainage of any remaining blood from the mediastinum. The subject is usually transferred out of intensive care the day after surgery.

Some subjects, however, develop myocardial dysfunction and low cardiac output syndrome (LCOS) during and after cardiac surgery. LCOS contributes to subjects' morbidity and mortality, as well as increasing length of stay and hospital costs.

To reduce or eliminate the development of myocardial dysfunction in a subject following cardiac surgery, one or a combination of cardiac drugs is administered to the subject at 14. Non-limiting examples of cardiac drugs include those listed above, as well as β-blockers, inotropic agents, chronotropic agents, preload reduction agents, and calcium channel antagonists. Examples of positive inotropic drugs are known in the art and include, but are not limited to, calcium, calcium sensitizers, cardiac glycosides, catecholamines, eicosanoids, and phosphodiesterase inhibitors. Non-limiting examples of catecholamines include dopamine, dobutamine, dopexamine, epinephrine, isoprenaline and norepinephrine.

The administration route and dosage of the cardiac drug will depend on the type of cardiac surgery being performed, as well as a number of other factors. For example, the dosage will depend upon the age, health, and weight of the subject, kind of concurrent treatment, if any, frequency of the treatment, and the nature of the effect desired. Examples of administration routes include parenteral (e.g., intravenous, intradermal, subcutaneous), oral, transdermal (topical), and transmucosal.

In one example of the present invention, a subject having undergone CABG may develop LCOS following surgery and thus require administration of a positive inotropic drug to improve cardiac contractility. At 14, a positive inotropic drug, such as dobutamine can be administered to the subject to improve cardiac contractility. Using an intravenous approach, for example, dobutamine can be administered to the subject in an amount effective to increase cardiac contractility. For instance, dobutamine can be administered to the subject at about 0.5 μg/kg/min to about 50 μg/kg/min using an intravenous drip line (not shown).

Administration of the cardiac drug following cardiac surgery, however, can cause undesirable cardiac rhythm disturbances, such as temporary atrial fibrillation, ventricular fibrillation, bradycardia, or combinations thereof. For example, administering dobutamine following cardiac surgery can lead to the development of sinus tachycardia. To minimize or eliminate cardiac drug-related rhythm disturbances, while still benefiting from the increased cardiac output associated with such drugs, an electric current can be delivered to a cardiac fat pad 36 of the subject at 16.

Electric current is delivered to a cardiac fat pad 36 of the subject to modulate vagal activity and thereby mitigate or eliminate the cardiac rhythm disturbance. Electric current is applied to a cardiac fat pad 36 using any one or combination of known electrostimulatory devices (not shown). Generally, the electrostimulatory device can include any device capable of providing selective cardiac fat pad 36 stimulation to pace the heart 20, improve contractility, and thus provide a stimulus to improve pumping efficiency and cardiac output. For example, the electrostimulatory device can include an electrical lead (not shown) having at least one electrode (not shown) operably connected thereto. The electrode can have any shape and size including, for example, a triangular shape, a rectangular shape, an ovoid shape, and/or a band-like shape (e.g., a split band configuration). For example, the electrode can have any size from about 5 degrees to about 360 degrees, and may be wedge-shaped, pointed, rounded, etc. The electrode can comprise a ½ or ¼ ring configuration, a plate electrode, a paddle electrode, a cuff electrode, a cylindrical electrode, or the like, and be made of any material capable of conducting an electrical current, such as titanium, platinum, platinum-iridium, or the like.

The electrode may be configured so that the electrostimulatory device has a unipolar construction using surround tissue as a ground or, alternatively, a multipolar construction using leads connected to a portion of the electrostimulatory device. For example, multipolar electrical leads can be used to deliver electric current to a cardiac fat pad 36. Electrical leads can include epicardial leads and intravascularly-fed leads. As described in more detail below, electrical leads may be designed and positioned to provide multiple electrostimulatory effects, sensing, and pacing, for example, in addition to neural stimulation.

To facilitate focal delivery of electric current to a cardiac fat pad 36, the size and shape of the electrode may be varied as needed. Additionally or optionally, the entire surface area of the electrode may be conductive or, alternatively, only a portion of the surface area of the electrode may be conductive. By modifying the size, shape, and conductivity of the surface of the electrode, the surface area of the electrode that contacts a cardiac fat pad may 36 be selectively modified to facilitate focal delivery of electric current. For example, electric current can be delivered to the electrode such that the electric current is conducted only through selective portions of the electrode. Delivery of electric current can then be selectively controlled or “titrated” to achieve a desired physiological effect.

Electric current can be delivered to the electrostimulatory device using a variety of internal, passive, or active energy delivery sources (not shown). The energy delivery source may include, for example, radio frequency energy, X-ray energy, microwave energy, acoustic or ultrasound energy, such as focused ultrasound or high intensity focused ultrasound energy, light energy, electric field energy, thermal energy, magnetic field energy, combinations of the same, or any other energy delivery source used with implantable pulse generators known in the art. The energy delivery source can be directly or indirectly (e.g., wirelessly) coupled to the electrostimulatory device.

Electric current can be delivered to the electrostimulatory device continuously, periodically, episodically, or a combination thereof. For example, electric current can be delivered in a unipolar, bipolar, and/or multipolar sequence or, alternatively, via a sequential wave, charge-balanced biphasic square wave, sine wave, or any combination thereof. Where a plurality of electrodes are included as part of the electrostimulatory device, electric current can be delivered to all the electrodes at once or, alternatively, to only a select number of desired electrodes using a controller (not shown) and/or known complex practice, such as current steering.

The particular voltage, current, and frequency delivered to the electrostimulatory device may be varied as needed. For example, electric current can be delivered to the electrostimulatory device at a constant voltage (e.g., at about 0.1 v to about 25 v), at a constant current (e.g., at about 25 microampes to about 50 milliamps), at a constant frequency (e.g., at about 5 Hz to about 10,000 Hz), and at a constant pulse-width (e.g., at about 50 μsec to about 10,000 μsec).

The electrostimulatory device can be part of an open- or closed-loop system. In an open-loop system, for example, a physician may, at any time, manually or by the use of pumps, motorized elements, etc. adjust treatment parameters such as pulse amplitude, pulse width, pulse frequency, or duty cycle. Alternatively, in a closed-loop system, electrical parameters may be automatically adjusted in response to a sensed symptom or a related symptom indicative of the extent of the condition being treated. In a closed-loop feedback system, a sensor (not shown) that senses a condition (e.g., a metabolic parameter of interest, such as vagal activity) of the body can be utilized. More detailed descriptions of sensors that may be employed in a closed-loop system, as well as other examples of sensors and feedback control techniques that may be employed are disclosed in U.S. Pat. No. 5,716,377, which is hereby incorporated by reference in its entirety.

The electrostimulatory device can be directly or indirectly contacted with one or more cardiac fat pads 36, depending on the type of cardiac surgery being performed and/or the desired physiological effect. By “direct” it is meant that the electrostimulatory device is brought into physical contact with a cardiac fat pad 36. For example, where a subject is undergoing an open-chest procedure, the electrostimulatory device can be contacted directly with a cardiac fat pad 36. By “indirect” it is meant that the electrostimulatory device is positioned about a cardiac fat pad 36, without directly contacting the cardiac fat pad, such that delivery of electric current to the electrostimulatory device can modulate vagal activity. For example, an intravenously-fed lead can be positioned proximate to the SN fat pad 54 in a blood vessel, such as the right pulmonary artery 46 or the right pulmonary vein 44. Alternatively, an intravascularly-fed lead adapted to puncture through a vessel wall can be placed proximate to a cardiac fat pad 36 and then manipulated to pierce the vessel wall and directly contact the fat pad.

Electric current is applied to a cardiac fat pad 36 to modulate vagal activity in the subject. As used herein, the terms “modulate” or “modulating” refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, thermal, ultrasonic, optical or chemical, or a combination of two or more of these. The terms “modulate” or “modulating” can also be used to refer to a masking, altering, overriding, or restoring of neuronal activity.

Delivery of electric current to a cardiac fat pad 36 increases vagal activity and thereby activates parasympathetic efferents. Because fat pad ganglia form part of the parasympathetic efferent pathway, stimulation of cardiac fat pads 36 directly affects cardiac tissue. For example, stimulating the parasympathetic efferents can selectively affect cardiac rate, AV conduction, and contractility. Stimulation of the SVC-AO fat pad 48, for example, reduces contractility of the left ventricle 28 and, thus, can provide a treatment for diseases such as heart failure and/or post-myocardial infarction remodeling. Additionally, stimulation of the IVC-LA fat pad 56 can increase AV conduction and thereby affect timing between contractions in the right atrium 22 and the right ventricle 24.

In an example of the present invention, electric current is delivered to a SN fat pad 54 of a subject having undergone CABG surgery and suffering from LCOS following dobutamine therapy. Following dobutamine administration, an electrical lead is placed in direct contact with the SN fat pad 54. An electric current is then delivered to the electrode of the electrostimulatory device with an intensity of about 1 mA to about 5 mA, a pulse duration of about 0.1 ms to about 1 ms, and a frequency between about 20 Hz to about 30 Hz. To avoid or minimize tissue polarization, the direction of current flow through the electrode can be alternated.

Stimulation of the SN fat pad 54 provides direct vagal inhibition of the SA node 30, resulting in slowing of the sinus rate without prolonging SN conduction time. Thus, stimulation of the SN fat pad 54 eliminates or decreases sinus tachycardia, without directly affecting ventricular contractility, while also preserving the hemodynamic benefits of dobutamine (e.g., increased systolic blood pressure, increased diastolic blood pressure, increased left ventricular systolic blood pressure, and increased cardiac output). After the sinus rate has reached a desired point, the electrical lead is removed from the subject and the cardiac surgery is completed.

The following example is for the purpose of illustration only and is not intended to limit the scope of the claims, which are appended hereto.

### Example 1

A study was done to assess the effects of dobutamine administration followed by vagal stimulation on sinus tachycardia. The study was done using 4 open-chest canines. Dobutamine was infused at a rate of 2.5-7.5 μg/kg/min to increase the cardiac contractility and the sinus rate at least 30 beats per minute. Vagal nerve stimulation was applied at the epicardial sinus node fat pad, located at the junction of the right pulmonary vein and the right atrium. Vagal stimulation was done with an electrical lead comprising a silicone plate with four platinum electrodes imbedded therein. Rectangular current pulses were delivered with intensity in the range of 1 to 5 mA, duration of pulse in the range of 0.1 ms to 1 ms, and frequency of repetition of the pulses between 20-30 Hz. To avoid tissue polarization, the polarity of consecutive pulses was alternated. Vagal nerve stimulation was administered to slow the sinus rate to the control level. Hemodynamic data was collected and compared in canines during control, during administration of dobutamine, and during both dobutamine and vagal stimulation.

The results of the study are shown in Table 1.

TABLE 1
Administration of dobutamine followed by vagal
stimulation to treat sinus tachycardia

| | | | Dobutamine + Vagal |
|---|---|---|---|
| | Control | Dobutamine | Stimulation |
| RR interval | 559 ± 27 | 421 ± 36* | 560 ± 16 |
| (ms) | | | |
| SBP (mmHg) | 99 ± 4 | 135 ± 18* | 126 ± 12* |
| DBP (mmHg) | 56 ± 5 | 84 ± 7* | 74 ± 7* |
| LVSP (mmHg) | 96 ± 7 | 130 ± 13* | 120 ± 16* |
| LVEDP | 7.2 ± 2.1 | 6.9 ± 2.2 | 7.6 ± 1.5 |
| (mmHg) | | | |
| +dp/dt | 1359 ± 206 | 3607 ± 861* | 2980 ± 602* |
| (mmHg/s) | | | |
| −dp/dt | −1351 ± −170 | −2259 ± −495* | −1897 ± −313* |
| (mmHg/s) | | | |
| CO (L/min) | 2.7 ± 0.1 | 4.2 ± 0.9* | 3.6 ± 0.8* |

*p < 0.05 vs. control

As shown in Table 1, dobutamine administration significantly increased heart rate, systolic blood pressure (SBP), diastolic blood pressure (DBP), left ventricular systolic pressure (LVSP) (and its ±dp/dt), and cardiac output (CO). Combining vagal stimulation with dobutamine eliminated sinus rate increase while preserving all other hemodynamic benefits.

From the above description of the invention, those skilled in the art will perceive improvements, changes and modifications. It will be appreciated that the order of steps illustrated in FIG. 1 and described above can be varied as needed. For example, administration of the cardiac drug can be performed simultaneous with cardiac surgery or, alternatively, administration of the cardiac drug can be performed simultaneous with application of electric current to a cardiac fat pad 36. Such improvements, changes, and modifications are within the skill of the art and are intended to be covered by the appended claims.

Having described the invention, we claim:

1. A method for treating a subject, the method comprising the steps of:
performing cardiac surgery on the subject;
administering a cardiac drug to the subject; and
applying an electric current to a cardiac fat pad of the subject to reduce an adverse effect caused by the cardiac drug.

2. The method of claim 1, wherein the adverse effect includes a cardiac rhythm disturbance.

3. The method of claim 2, wherein the cardiac rhythm disturbance includes sinus tachycardia.

4. The method of claim 1, wherein the cardiac drug comprises a positive inotropic drug.

5. The method of claim 4, wherein the positive inotropic drug is selected from the group consisting of calcium, a calcium sensitizer, a cardiac glycoside, a catecholamine, an eicosanoid, and a phosphodiesterase inhibitor.

6. The method of claim 5, wherein the catecholamine is selected from the group consisting of dopamine, dobutamine, dopexamine, epinephrine, isoprenaline and norepinephrine.

7. The method of claim 1, wherein said step of applying an electric current to a cardiac fat pad includes applying an electric current to a sinus node fat pad.

8. The method of claim 1, wherein said step of administering a cardiac drug is performed simultaneous with said step of performing cardiac surgery on the subject.

9. The method of claim 1, wherein said step of administering a cardiac drug is performed simultaneous with said step of applying an electric current to a cardiac fat pad of the subject.

10. The method of claim 1 further comprising the step of configuring the electric current so as to increase vagal activity in the subject.

11. A method for treating a subject, said method comprising the steps of:
performing cardiac surgery on the subject;
administering a positive inotropic drug to the subject;
applying an electric current to a sinus node fat pad of the subject; and
configuring the electric current so as to increase vagal activity in the subject and thereby reduce sinus tachycardia caused by the positive inotropic drug.

12. The method of claim 11, wherein the positive inotropic drug is selected from the group consisting of calcium, a calcium sensitizer, a cardiac glycoside, a catecholamine, an eicosanoid, and a phosphodiesterase inhibitor.

13. The method of claim 12, wherein the catecholamine is selected from the group consisting of dopamine, dobutamine, dopexamine, epinephrine, isoprenaline and norepinephrine.

14. The method of claim 11, wherein said step of administering a positive inotropic drug is performed simultaneous with said step of performing cardiac surgery on the subject.

15. The method of claim 11, wherein said step of administering a positive inotropic drug is performed simultaneous with said step of applying an electric current to a sinus node fat pad of the subject.
